(19) United States
(12) Patent Application Publication
(10) Pub. No.: US 2003/0096774 A1
(43) Pub. Date: May 22, 2003

(54) Compositions of nucleic acids and cationic aminoglycosides and methods of using and preparing the same

(76) Inventors: Igor Gonda, San Francisco, CA (US); Deepa Deshpande, Fremont, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 09/990,181

(22) Filed: Nov. 21, 2001

(51) Int. Cl. A61K 48/00; A61K 31/704; A61L 9/04

(52) U.S. Cl. 514/44; 514/37; 514/39; 424/46

(57) ABSTRACT

Compositions that include nucleic acid and cationic aminoglycosides and methods for their use are provided. The subject compositions are characterized by having nucleic acid complexed with a cationic aminoglycoside, where the nucleic acid is condensed. In certain embodiments, the cationic aminoglycoside is a cationic aminoglycoside antibiotic. The composition may further include one or more of: functional groups such as targeting moieties, nuclear localization or targeting peptides, endosomolytic peptides and/or one or more lipids and/or polymers, where the lipids may be provided in a manner to encapsulate the nucleic acid. The present invention also provides methods of using and preparing the nucleic acid-aminoglycoside compositions.

### GOVERNMENT RIGHTS

The United States Government may have certain rights in this application pursuant to Grant 5-R44-CA81660-03 from the National Institute of Health.

### FIELD OF THE INVENTION

The field of the invention is generally directed toward the use of cationic species for complexing with nucleic acids and more particularly the use of cationic aminoglycosides for complexing with nucleic acids. Such complexes may be used for the introduction of nucleic acids and/or gene products into cells.

### BACKGROUND OF THE INVENTION

A number of methods have been used for delivery and expression of foreign genes in vitro and in vivo. These include chemical methods (calcium phosphate precipitation, DEAE-dextran, neutral or anionic liposomes, cationic species such as cationic liposomes and targeted polylysine conjugates etc.), physical methods (microinjection, electroporation and biobalistics) and biological methods (viral vectors) (Felgner (1993) J. Liposome Res., 3:3-16).

Practically speaking, an ideal gene delivery vector should have the following characteristics: (1) it should protect and deliver DNA into cells efficiently and effectively, preferably with specificity toward a particular cell type; (2) it should be non-toxic; and (3) it should be easy to produce in large quantity. Current vector systems do not adequately meet all these requirements.

Adenovirus, for example, is a highly efficient vector for gene transfer and can transiently infect cells of different types. Engineered adenovirus was believed to be relatively safe for the host (Rosenfeld et al. (1992) Cell, 68:143-155; Engelhardt et al. (1994) Proc. Natl. Acad. Sci. USA, 91:6196-6200) and, compared with other recombinant viral vectors, adenovirus is relatively easy to produce in large quantity. However, recent preclinical and clinical trials have raised serious concerns about its immunogenicity. Treatment related inflammation, production of neutralizing antibodies and virus specific cytotoxic T lymphocyte (CTL) response in the host may prevent this viral vector from being used at high doses or administered repeatedly (Crystal et al. (1994) Nature Genetics, 8:42-51).

Retrovirus and adeno-associated virus (AAV) mediate efficient and stable transfection to dividing and possibly nondividing cells (Miller (1990) Hum Gene Ther, 1:5; Kotin (1994) Hum Gene Ther, 5:793-801). However, relatively low viral titers have been the major technical limitation for both systems.

As mentioned above, cationic species have been used in attempts to stabilize, package and increase the transfection of nucleic acids. Such methods include condensing nucleic acid with cations, usually polycations such as polyamines. Cationic liposomes and targeted polylysine conjugates have also been explored (Felgner et al. (1991) Nature, 349:351-352; Curiel et al. (1991) Proc Natl Acad Sci USA, 88:8850-8854). However, although many cationic based methods exist, they suffer from a number of critical deficiencies, including toxicity, immunogenicity and lack of targeting ability.

As such, a need exists to provide improved nucleic acid/gene product compositions and methods for using the same for the delivery of such compositions into targeted cells. Of interest would be the development of such compositions that are safe, i.e., therapeutically non toxic (i.e., the subject compositions have therapeutically acceptable levels of toxicity), can effectively condense nucleic acids, can achieve high transfection efficiencies and which may provide additional antimicrobial therapy. This need and others are addressed by the instant invention.

### SUMMARY OF THE INVENTION

Compositions that include nucleic acid and cationic aminoglycosides and methods for their use are provided. The subject compositions are characterized by having nucleic acids complexed with a cationic aminoglycosides, where the nucleic acid is condensed. In certain embodiments, the cationic aminoglycoside is a cationic aminoglycoside having bacteriostatic or bactericidal effects. A variety of cationic aminoglycosides are suitable for use with the present invention, where representative cationic aminoglycosides include, but are not limited to, cationic aminoglycosides such as gentamicin, tobramycin, amikacin, streptomycin, neomycin, sisomicin and netilmicin. The subject composition is characterized at least by (1) therapeutic non toxicity (i.e., therapeutically acceptable levels of toxicity) (2) therapeutically acceptably levels of immunogenicity, (3) transfection rates sufficiently sufficient to carry out effective therapeutic treatment, (4) ease of manufacture and (5) minimal manufacturing cost. The composition may further include one or more of: functional groups such as targeting moieties, nuclear localization or targeting peptides, endosomolytic peptides and/or one or more lipids and/or polymers, i.e., therapeutically acceptable lipid(s) and/or polymers, provided in a manner so as to interact with the nucleic acid and thus promote transfection. The subject compositions may be delivered or administered to a subject or cell using a variety of means, including, but not limited to pulmonary, parenteral (i.e., intravenous, intramuscular, subcutaneous, intratracheal), oral, nasal, intraperitoneal, intraocular, intracranial, suppository, dermal, transdermal and buccal. The present invention also provides methods of using and preparing the nucleic acid-aminoglycoside complexes or compositions.

Thus, the nucleic acid-aminoglycoside complexes or compositions of the subject invention provides a means for introducing a nucleic acid and/or a gene product into a cell, thereby providing a method for administering the product to the cell for a variety of purposes. These delivery devices can be administered as a pharmaceutical formulation, i.e., with an excipient carrier.

Although the above-mentioned nucleic acid-aminoglycoside complexes can be administered via any conventional route, in many embodiments they are aerosolized and delivered via a pulmonary route.

It is an aspect of the invention to provide a nucleic acid composition that does not generate a therapeutically unacceptable immune response.

It is yet another aspect of the invention to provide a nucleic acid composition that is therapeutically non toxic, i.e., the subject compositions have therapeutically acceptable levels of toxicity.

It is yet another aspect of the invention to provide a nucleic acid composition that has a transfection efficiency sufficiently robust to provide for clinically effective therapies.

It is yet another aspect of the invention to provide a nucleic acid composition that can also provide bacteristatic or bactericidal effects.

It is an advantage that the nucleic acid formulation is non toxic, i.e., the subject compositions have therapeutically acceptable levels of toxicity.

It is yet another advantage that the interaction between the nucleic acid and the cationic aminoglycoside provides a condensed nucleic acid with increased transfection efficiency.

It is yet another advantage that the nucleic acid complexes of the subject invention provide bacteriostatic or bactericidal therapy

These and other aspects, advantages and features of the present invention will become apparent to those persons skilled in the art upon reading the details of the presently described invention.

### BRIEF DESCRIPTION OF THE DRAWINGS

FIG. 1 is a graph depicting the effect that DNA complexed with the aminoglycoside antibiotic Gentamicin has on transfection relative to transfection with naked DNA.

### DEFINITIONS

The terms “nucleic acid” or “polynucleotide” as used herein are considered interchangeable unless otherwise indicated, and encompass DNA, RNA, and/or DNA or RNA with altered backbone and/or bases modified from native or any mixture thereof. Nucleic acids according to the present invention may also include any strand structure, e.g., single-, double- or triple-stranded polynucleotide structures or mixtures thereof. Also, the nucleic acids may comprise a linear or circular structures, e.g., plasmids, phagemids, cosmids, etc. the nucleic acids of the subject invention may be naturally occurring, synthetically or semi-synthetically produced.

The term “gene product” as used herein refers an oligopeptide, peptide, or protein generated from a nucleic acid introduced into or to a cell using the methods of the present invention.

The terms “treatment”, “treating” and the like are used herein generally to mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. In one embodiment, “treatment” as used herein covers any treatment of a disease in a mammal, particularly a human, and includes:

(a) preventing the disease or its symptoms from occurring in a subject which may be predisposed to the disease but has not yet been diagnosed as having it;

(b) inhibiting the disease or its symptoms, i.e., arresting its development; or

(c) relieving the disease, i.e., causing regression of the disease or its symptoms. In another embodiment, the term “treatment” as used herein covers any use for inhibiting or enhancing a normal biological process.

The terms “cationic aminoglycoside” and “polycationic aminoglycoside” used herein interchangeably, herein refers to a positively charged macromolecule that includes a carbohydrate having at least one amine group, including both known or as yet unidentified cationic aminoglycosides. The cationic aminoglycosides for use in the present invention may vary in size, but typically have an average molecular weight ranging from about 300 to about 800 Daltons. The cationic aminoglycosides of the present invention are polycationic at physiological pH, are basic, water soluble molecules and may also possess bacteriostatic or bactericidal capabilities, i.e., they are able to destroy or inhibit the growth of bacteria, fungi, or other harmful microorganisms without damaging the host, e.g., aminoglycoside antibiotics. Cationic aminoglycosides suitable for use with the subject invention include, but are not limited to, Gentamicin, Tobramycin, Amikacin, Streptomycin, Neomycin, Sisomicin and Netilmicin.

The term “condensed” as used herein refers to the phenomenon whereby nucleic acid, e.g., DNA molecules interact electrostatically with polyvalent cationic species with about 10³ to about 10⁶ reduction (random coil-compact particle transition) in the physical volume of the nucleic acid, e.g., of the random coil of DNA. Polyvalent cationic species can not only condense electrostatically on DNA, but can also cause the collapse of the tertiary structure of DNA when more than 90% of the charge is neutralized. Such DNA, i.e., DNA condensed with polyvalent cationic species, is less susceptible to degradation by nucleases.

### DETAILED DESCRIPTION OF THE SPECIFIC EMBODIMENTS

Before the present invention is described, it is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “an”, and “the” include plural referents unless the context clearly dictates otherwise.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### GENERAL ASPECTS OF THE INVENTION

The present invention provides compositions and efficient methodologies to effectively tranfect complexes of nucleic acids and cationic aminoglycosides into target cells. High transfection efficiencies are achieved by the subject methods due at least in part to the condensed configuration of the nucleic acid when present in such a complex. The cationic aminoglycosides used in the present invention may be any convenient cationic aminoglycoside, where cationic aminoglycosides having bacteriostatic or bactericidal capabilities are of particular interest. It will be apparent to those of skill in the art that when complexed with nucleic acid, the use of such cationic aminoglycosides having bacteriostatic or bactericidal capabilities advantageously accomplishes both nucleic acid condensation which increases transfection and may further act as a therapeutic agent, in addition to the therapeutic effect derived from the complexed nucleic acid. The nucleic acid can either prevent expression of an endogenous protein (e.g., an antisense oligonucleotide) or will encode and express any protein, preferably a therapeutic gene product.

The nucleic acid-cationic aminoglycoside complexes of the present invention can thus be used as pharmacological agents targeted to cells. These complexes have numerous advantages for introducing nucleic acids and their resulting gene products into cells in vivo.

First and foremost, these complexes or compositions may increase transfection efficiencies, relative to naked DNA, by as much as about 100% or more and allow dose reduction. Typically, transfection efficiencies, relative to naked DNA, may be increased by as much as about 100% to about 200%, and may be increased by as much as about 500% to about 1,000% or more. Accordingly, the subject complexes may increase transfection efficiencies, relative to transfection efficiencies of naked DNA, by about 100% to 1000% or more, depending on the particular aminoglycoside used. The cationic aminoglycosides facilitate condensation of the nucleic acids, which increases the stability to nucleases, facilitates interaction with the predominantly negatively charged cell surface and ultimately the transfection thereof. This results in the increase in nucleic acid concentration at the target cells, which enables dose reduction.

Second, in those embodiments using conventional cationic aminoglycosides, e.g., aminoglycosides known to exhibit minimal or no toxicity or therapeutically acceptable levels of toxicity, the toxicity of the subject complexes is, consequently, also minimized or exhibits therapeutically acceptable levels. For example, certain aminoglycosides have demonstrated therapeutically acceptable levels of toxicity or no toxicity such that at particular dosages they are tolerated by a human, where these certain aminoglycosides are suitable for use in the subject invention at therapeutically acceptable levels. For example, tobramycin aerosols are approved by the U.S. Food and Drug Administration for administration by inhalation for the treatment of pulmonary infections in cystic fibrosis, where the administered dose is 300 mg. Similarly, it is known that certain aminoglycosides may be delivered intravenously and subcutaneously at high doses without adverse or unacceptable therapeutic effects, e.g., gentamicin: 5 mg/kg of body weight per day; amikacin: 15 mg/kg of body weight/day in patients with normal renal function. Therefore, the use of aminoglycosides for DNA delivery affords high doses without the concomitant toxicity of other polycationic molecules (see for example Physicians' Desk Reference, 2001).

Third, where cationic aminoglycosides having bacteriostatic or bactericidal effects are used to complex with the nucleic acid, the nucleic acid-cationic aminoglycoside complexes are thus capable of acting therapeutically both by means of the transfected nucleic acid and the aminoglycoside employed.

Fourth, the transfection efficiencies may be further increased by incorporating or including lipids and/or polymeric molecules into the subject compositions.

Fifth, the subject compositions may be delivered or administered to a subject or cell using a variety of means or routes, including, but not limited to, pulmonary, parenteral (i.e., intravenous, intramuscular, subcutaneous, intratracheal), oral, nasal, intraperitoneal, intraocular, intracranial, suppository, dermal, transdermal and buccal.

These and other advantages of the present invention overcome many of the shortcomings of conventional non-viral nucleic acid delivery methods. In addition, the delivery systems do not have many of the shortcomings associated with physical or viral means of introduction of nucleic acids and gene products into cells in vivo.

### NUCLEIC ACID-AMINOGLYCOSIDE COMPLEXES

As described above, the present invention provides complexes that include nucleic acids and cationic aminoglycosides such that the nucleic acids are condensed when combined with the aminoglycosides. The nucleic acids which can be complexed or otherwise associated with the aminoglycosides according to the present may include sense or antisense polynucleotides. For example, antisense oligonucleotides used may selectively inhibit the expression of target DNAs. For example, antisense oligonucleotides may be complexed with a cationic aminoglycoside, where such antisense oligonucleotides are complementary to viral sequences and utilized for antiviral treatments, e.g., hepatitis, AIDS viral infection, papillomavirus infection, etc. The use of antisense oligonucleotides for genetic therapy has been reported in the literature (See, for example, Stein and Chang,(1993) Science 261: 1004). Also, ribozymal RNAs may be complexed with a cationic aminoglycoside and used to study gene expression or for genetic therapy.

In one embodiment of the subject invention, the present invention provides for the efficient complexing of high molecular weight (“HMW”) polynucleotide molecules. As used herein, “high molecular weight” polynucleotide refers to a polynucleotide molecule that comprises at least one coding sequence that can be transcribed when the polynucleotide is introduced into a host cell. This transcription can produce an mRNA molecule that can then be translated to produce a polypeptide or protein, or it can produce an antisense RNA molecule. Transcription of the coding sequence of the HMW polynucleotide is preferably under the control of cis-acting regulatory elements, such as enhancer sequences, operator sequences and the like, and the polynucleotide also contains a ribosome binding site, an initiation codon and transcription termination and polyadenylation signals. The definition of HMW polynucleotides as used herein is, therefore, generally understood to mean polynucleotides that contain such regulatory elements. The HMW polynucleotide may also contain other elements such as origins of replication as are commonly found on polynucleotides used for transfection.

The present invention provides for the efficient complexing of large vectors, including those which have operably integrated therein sequences that permit stable, episomal maintenance and those which encode multigene cassettes. This is significant, in the case of episomal constructs, because integration of the desired nucleic acid into the host cell's genome may have a negative impact on the transfection process. For multigene cassettes, it also is important as coordinate regulation of the encoded genes can be more easily achieved.

The nucleic acids which may suitable for use with the present invention may range in size from as small as about 10 bases to about 100 kilobases or longer, usually about 10 bases to about 50 kilobases and more usually about 3 kilobases to about 15 kilobases.

In many embodiments of the subject invention, the nucleic acids will include an episomal element, e.g., a plasmid, which contains one or more genes which are to be expressed in target cells. An episomal element containing an origin of replication that is recognized by the replication functions of the host cell will be stably maintained in the cell as an extrachromosomal element, thereby allowing stable expression of genes encoded on the element. In general, these genes will cause the target cell to produce a heterologous expression product, or acquire an altered phenotype. If the episomal element does not contain an origin or replication that is recognized by the host cell, the expression product will be produced only transiently.

As described above, the nucleic acid of the present invention may comprise DNA, RNA or a mixture thereof, and may comprise linear or circular structures. Also, the nucleic acids may be single or multi-stranded and may include sense or antisense nucleic acid sequences. In many embodiments, the nucleic acids will include DNA constructs having a size ranging from about 10 bases to about 100 kilobases or longer, usually about 10 bases to about 50 kilobases and more usually about 3 kilobases to about 15 kilobases, as mentioned above. In general, such DNA constructs will contain a gene or genes which are to be expressed in the targeted cells. The DNA construct may also contain suitable regulatory sequences which provide for the expression of these genes, in addition to sequences that provide for these DNA constructs to autonomously replicate in target cells if necessary, and also suitable selectable markers. In general, these genes will be expressed under the control of regulatable promoters.

The DNA constructs of the present invention will typically contain a gene or genes which produce a therapeutic or desired gene product. Examples of such gene products include, but are not limited to, therapeutic lymphokines, cytokines, hormones, cell adhesion molecules, enzymes or enzyme inhibitors, receptors, ion channels, transcription factors, protein kinases, protein phosphatases, and cellular antigens for generating an immune response in a host. Alternatively the DNA constructs will contain suicide genes, tumor suppressor genes, genes encoding antisense RNAs, or genes that induce or prevent cellular apoptosis.

Examples of lymphokines and cytokines that can be encoded by the aminoglycoside complexed DNA constructs of the present invention include, but are not limited to, platelet-derived growth factor, epidermal growth factor, interleukins 1-14, granulocyte colony stimulating factor, granulocyte-macrophage colony stimulating factor, tumor necrosis factor, leukemia inhibitory factor, amphiregulin, angiogenin, betacellulin, calcitonin, ciliary neurotrophic factor, brain-derived neurotrophic factor, neurotrophins 3 and 4, nerve growth factor, colony stimulating factor-1, endothelial cell growth factor, erythropoietin, acidic and basic fibroblast growth factor, hepatocyte growth factor, heparin binding EGF-like growth factor, insulin, insulin-like growth factors I and II, interferons .alpha., .beta., and .gamma., keratinocyte growth factor, macrophage inflammatory protein .alpha. and .beta., midkine, oncostatin M, RANTES, stem cell factor, transforming growth factors .alpha. and .beta., and vascular endothelial growth factor. Examples of cell adhesion molecules include integrins, cadherins, selecting, and adhesion molecules of the immunoglobulin superfamily, such as VCAM, ICAM, PECAM, and NCAM. Examples of tumor suppressor genes include p53, DCC, Rb, and MTS1. Those of skill in the art will recognize that other genes can also be used in the subject invention.

In addition, the DNA constructs will typically include regulatory elements that can control replication of the construct within the cell, as well as transcription and translation of genes encoded on the construct. For use in in vivo delivery of nucleic acids, it is sometimes useful for these regulatory elements to be tissue specific. The term “tissue-specific promoter” or “tissue-specific transcriptional regulatory sequence” indicates a transcriptional regulatory sequence, promoter and/or enhancer that is induced selectively or at a higher level in cells of the target tissue than in other cells. For example, tumor cell-specific promoters include promoters that are induced selectively or at a higher level in a particular cell type or a tumor cell. Tissue specific promoters are known in the art, where examples include, but are not limited to. the alpha-actin promoter (Shani (1986), Mol. Cell. Biol., 6:2624); the elastase promoter (Swift et al.(1984), Cell, 38:639); the alpha-fetoprotein promoter (Krumlauf et al.(1985), Nature, 319:224-226); the beta-globin promoter, (Townes et al.(1985), EMBO J., 4:1715); the human growth hormone promoter (Behringer et al.(1988), Genes Dev., 2:453); the insulin promoter (Selden et al.(1986), Nature, 321:545) and a prostate-specific promoter (Allison et al.(1989), Mol. Cell. Biol., 9:2254).

Regardless of the type of nucleic acid employed in the present invention, the nucleic acid is combined or complexed with a cationic aminoglycoside, as described above. As is known to those of skill in the art, most aminoglycosides are polycationic at physiological pH, i.e., are positively charged macromolecules at pH ranging from neutral to acidic and thus the optimum effective range of the subject compositions are such. The cationic aminoglycosides of the present invention typically have a molecular weight in the range from about 300 to 800 Daltons and may be naturally occurring or synthetically or semi-synthetically produced.

As described, the cationic aminoglycosides employed in the present invention are combined with the nucleic acid of interest. That is, the aminoglycoside and nucleic acid form a stable complex wherein the complexed nucleic acid is condensed when combined with the cationic aminoglycoside. The cationic aminoglycosides of the subject formulation interact with nucleic acid through the electrostatic interaction of the negative charges of the nucleic acids and the positive charges of the aminoglycoside. This nucleic acid/aminoglycoside complex thus facilitates the delivery of functional nucleic acid into the cells.

The amount of nucleic acid to aminoglycoside will vary according to a variety of factors, including, but not limited to, the particular type of nucleic acid used, the particular type of aminoglycoside used, and the like. By way of example and not limitation, in certain embodiments of the subject invention, the ratio of nucleic acid to aminoglycoside ranges from about 1:0.001 to about 1:1000. This range of ratios is exemplary only and of course may vary as required.

Accordingly, the aminoglycosides employed are able to condense the nucleic acid, where the degree of condensation may vary depending on a variety of factors including, but not limited to, the particular nucleic acid and particular aminoglycoside used, the final pH, and the like. Typically, the nucleic acid is condensed from about 1000 fold to about 1,000,000 fold or more. In other words, the nucleic acid molecules, e.g., DNA, interact electrostatically with polyvalent cationic species with about 10³ to about 10⁶ reduction (random coil-compact particle transition) in the physical volume of the random coil of DNA. Polyvalent cationic species can not only condense electrostatically on DNA, but can also cause the collapse of the tertiary structure of DNA when more than 90% of the charge is neutralized. As described above, such DNA, i.e., DNA condensed with polyvalent cationic species, is less susceptible to degradation by nucleases.

In certain embodiments, the aminoglycosides of the present invention have bacteriostatic or bactericidal properties. In other words, the aminoglycosides of the subject invention are able to destroy or inhibit the growth of bacteria, fungi, or other harmful microorganisms without damaging the host, e.g., by inhibiting protein synthesis. Thus, the aminoglycosides employed in the present invention can be chosen with respect to a number of factors, including, but not limited to, the degree to which it complexes and condenses with the nucleic acid of interest and the therapeutic effectiveness of the aminoglycoside, typically for a particular disease state or infection. For example, where the nucleic acid is one which is chosen to provide gene therapy to an individual suffering from, for example, cystic fibrosis, aminoglycosides conventionally used to treat infections relating to cystic fibrosis may be used to complex with the nucleic acid such as gentamicin and tobramycin, thus providing a dual therapeutic methodology. In other words, gene therapy may be provided by the nucleic acid and bacteriostatic or bactericidal therapy may be provided by the aminoglycoside

A variety of cationic aminoglycosides may be used with the subject invention, where representative cationic aminoglycosides include, but are not limited to, gentamicin, tobramycin, amikacin, streptomycin, neomycin, sisomicin and netilmicin.

In certain embodiments of the subject invention, the formulation also includes one or more lipids or polymers for enhancing stability, potency and/or transfection, i.e., therapeutically acceptable lipids. The lipid(s) may be provided in a manner so as to form a vesicle or liposome which encapsulates the condensed nucleic acid (however, other forms of interaction are contemplated by this invention as well). Particularly, cationic liposomes are of particular interest where such cationic liposomes advantageously fuse with negatively charged cell surfaces and thus promote transfection. A variety of therapeutically acceptable lipids may be used to encapsulate the nucleic acid, where the particular lipid(s) is chosen with respect to a variety of factors that include, but are not limited to, the targeted cell, the particular nucleic acid/aminoglycoside complex, and the like.

For example, lipofectin (Gibco BRL, Gaithersburg, Md.) has been successfully used for the transfection of various cell lines in vitro (Felgner et al.(1987), Proc. Natl. Acad. Sci. U.S.A., 84:7413-7417). Lipofectin is formed with the cationic lipid DOTMA, N-[1-(2,3-dioleyloxy)propyl]-N,N,N-trimethylammonium chloride, and DOPE, dioleylphosphatidyl ethanolamine at a 1:1 molar ratio. The liposomes prepared with this formulation are thought to spontaneously interact with DNA through the electrostatic interaction of the negative charges of the nucleic acids and the positive charges at the surface of the cationic liposomes. This DNA/liposomal complex fuses with tissue culture cells and facilitates the delivery of functional DNA into the cells (Felgner et al., supra).

Behr et al.(1989), Proc. Natl. Acad. Sci. U.S.A., 86:6982-6986) and Barthel et al. (1993), Cell Biol, 12:553-560) have reported the use of a lipopolyamine (DOGS, Spermine-5-carboxy-glycinediotadecylamide) to transfer DNA to cultured cells. Lipopolyamines are synthesized from a natural polyamine spermine chemically linked to a lipid. For example, DOGS is made from spermine and dioctadecylamidoglycine (Behr et al., supra). DOGS spontaneously condense DNA on a cationic lipid layer and result in the formation of nucleolipidic particles. This lipospermine-coated DNA shows high transfection efficiency (Barthel et al., supra).

Cationic liposomes containing multivalent cationic lipid usually show better transfection activities than those containing monovalent lipids and thus are also of interest in the present invention (Behr et al.,(1989) Proc Natl Acad Sci USA, 86:6982-6986; Hawley-Nelson et al. (1993) Focus, 15:73-79). For example, LipofectAMINE (GIBCO BRL, Gaithersburg, Md., USA) is consistently more active in transfection than Lipofectin (GIBCO BRL) (Hawley-Nelson et al., supra) and as such may be employed with the present invention.

All cationic lipid molecules contain four different functional domains: a positively charged head group(s), a spacer of varying length, a linker bond and a hydrophobic anchor. The head group of most known cationic lipids contains a simple or multiple amine group with different degrees of substitution, with one exception being an amidine group (Ruysschaert et al., (1994), Biochem Biophys Res Commun, 179:280-285). The amine groups range from primary amine to quaternary ammonium with substitution of methyl or hydroxyethyl groups. In some cases several different types of amino groups coexist in a single cationic lipid (dioctadecyldimethylammonium chloride (DOGS) and 2,3-dioleoyloxy-N-(2(sperminecarboxamido)-ethyl)-N,N-dimethyl-1-propanamimium trifluoroacetate (DOSPA)) (Behr et al., supra; Hawley-Nelson et al., supra). The number of charged groups varies from monovalent to multivalent (Felgner et al. (1987), Proc Natl Acad Sci USA, 84:7413-7417; Felgner et al. (1994), J. Biol. Chem., 269:1550-1561; Behr et al., supra; Farhood et al. (1992), Biochim. Biophys. Acta., 1111:239-246; Gao et al. (1991), Biochim. Biophys. Res. Commun., 179:280-285; Zhou et al. (1991), Biochim. Biophys. Acta., 1065:8-14; Rose et al. (1991), Biotechniques, 10:520-525; Hawley-Nelson et al., supra; Ruysschaert et al., supra; Ito et al. (1990), Biochem. Intl., 22:235-241; Leventis et al. (1990), Biochem. Biophys. Acta., 1023:124-132; Guo et al. (1993), J. Liposome Res., 3:51-70; Akao et al. (1994), Biochem. Mol. Biol. Intl., 34:915-920). The head group of a cationic lipid is responsible for interactions between liposome and DNA, and between liposome-DNA complex and cell membrane or other components of the cell. The interaction is vital for the transfection activity and may contribute to the toxicity of the treatment.

Examples of lipids suitable for use in the invention include, e.g., known vesicle or liposome forming compounds such as phosphatidylcholine, both naturally occurring and synthetically prepared, phosphatidic acid, lysophosphatidylcholine, phosphatidylserine, phosphatidyl ethanolamine, sphingolipids, phosphatidylglycerol, sphingomyelin, cardiolipin, glycolipids, gangliosides, and cerebrosides such as soybean phospholipids. Other suitable lipids include steroids, cholesterol, aliphatic amines such as long chain aliphatic amines and carboxylic acids, long chain sulfates and phosphates, diacetyl phosphates, butylated hydroxy toluene, tocopherol, retinol and isoprenoid compounds which may confer desired properties to the formed liposomes.

Also, synthetic phospholipids containing either altered aliphatic portions such as hydroxyl groups, branched carbon chains, cyclo derivatives, aromatic derivatives, ethers, amides, polyunsaturated derivatives, halogenated derivatives or altered hydrophilic portions containing carbohydrate, glycol, phosphate, phosphamide, quaternary amines, sulfate, sulfonyl, carboxy, amine, sulphydryl, imidazole groups and combinations of such groups can be either substituted or intermixed with the above-mentioned lipids which may be used in the present invention. Lipids suitable for use in preparing liposomes are well known in the literature, and are described, e.g., in U.S. Pat. No. 4,201,767; U.S. Pat. No. 4,235,877; U.S. Pat. No. 4,241,046; U.S. Pat. No. 4,261,975; and U.S. Pat. No. 4,394,448, all of which are incorporated by reference in their entireties.

An active cationic liposome formulation is usually small, unilamellar liposomes prepared by sonication or microfluidization (Felgner et al. (1987) supra). Occasionally, multilamellar liposomes prepared by simple vortex (Felgner et al. (1994), supra), or dilution of lipid solution from ethanol solvent (Behr et al. (1989), supra), are also active. Cationic liposomes normally contain a cationic amphiphile and a neutral ‘helper’ lipid, dioleoylphosphatidylethanolamine (DOPE). DOPE is required for non-bilayer forming cationic lipids to form stable cationic liposomes; these include cationic cholesterol derivatives (Farhood et al. (1992), supra; Gao et al. (1991), supra), lipopolylysine (Zhou et al. (1991), supra), and some double-chain cationic surfactants (Rose et al. (1991), supra). Most double-chain cationic lipids can form liposomes by themselves, or form liposomes as a mixture with DOPE or other lipids.

The present invention embraces the use of one or more of any lipid, polymer or combination such as cationic polymers including lipids and dendrimers which provides the desired effect, i.e., a therapeutically acceptable or therapeutically effective effect, on potency and stability of the composition. In certain embodiments, the one or more lipids may be combined with other cationic polymers, e.g., cationic dendrimers/cationic lipids and poly-L-Lysine/cationic lipids, etc.

Following liposome preparation, the liposomes may be sized to achieve a desired size range using any convenient technique (see for example U.S. Pat. No. 4,737,323, incorporated herein by reference).

### USES OF THE NUCLEIC ACID-AMINOGLYCOSIDE COMPLEXES OF THE INVENTION

The condensed nucleic acid-aminoglycoside complexes will have many different potential uses, as will be apparent to those skilled in the art upon reading the present disclosure. For example, the nucleic acid-aminoglycoside complexes of the present invention can be used to produce cells or animals which express a defective gene or genes. The resulting cells or animals may be used as in vitro or in vivo models for assessing the efficacy of potential therapeutic agents.

A further utility for the nucleic acid-aminoglycoside complexes of the invention is for introducing into cells DNA or constructs that encode a therapeutic product or prevent transcription of an endogenous product. The therapeutic product can be, for example, an antisense RNA or ribozyme RNA molecule, or it can be a therapeutic protein. A “therapeutic protein” as used herein refers to a peptide, polypeptide, or protein that, when confers a therapeutic benefit to a host when administered to the host, or when it is expressed in cells of the host. The nucleic acid delivery can be in vivo, in which the nucleic acid-aminoglycoside complexes are introduced directly into a host animal, preferably a human, or can be ex vivo, in which isolated cells are first transfected with the nucleic acid-aminoglycoside complexes, and are then reintroduced into a host animal. Ex vivo nucleic acid delivery in humans is described in U.S. Pat. No. 5,399,346, which is hereby incorporated by reference in its entirety. See also Tolstoshev (1993), Annu. Rev. Pharmacol. Toxicol., 33:573-96, for a general review of nucleic acid delivery, which is also incorporated herein by reference in its entirety.

### PHARMACEUTICAL FORMULATIONS OF THE NUCLEIC ACID-AMINOGLYCOSIDE COMPLEXES OF THE SUBJECT INVENTION

The presently described nucleic acid-aminoglycoside complexes or compositions (which may or may not include one or more of: functional groups such as targeting moieties, nuclear localization or targeting peptides, endosomolytic peptides and/or one or more lipids/polymers, i.e., therapeutically acceptable lipid(s)/polymers, provided in a manner so as to form a vesicle or liposome which encapsulates the condensed nucleic acid and thus promotes transfection) may be administered to a subject by virtually any means used to administer conventional antibiotics. A variety of delivery systems are well known in the art for delivering bioactive compounds to an animal. The subject compositions may be delivered or administered to a subject or cell using a variety of means, including, but not limited to, pulmonary, parenteral (i.e., intravenous, intramuscular, subcutaneous, intratracheal), oral, nasal, intraperitoneal, intraocular, intracranial, suppository, dermal, transdermal and buccal administration. The specific delivery system used depends on the location of the area to be treated, and it is well within the skill of those in the art to determine the location and to select an appropriate delivery system. In certain embodiments of the subject invention, the nucleic acid-aminoglycoside complexes are delivered via pulmonary introduction, and oftentimes the nucleic acid-aminoglycoside complexes are administered to a patient in an aerosol inhalation device.

The aminoglycoside-nucleic acid complex, with or without other excipients such as phospholipids, can be delivered to the respiratory tract either as a liquid formulation, or as a dry powder, or as particles suspended in a liquid. A dry powder inhaler formulation may contain particles of appropriate aerodynamic size of inhalation into the deep lung (typically about 1-3 micron), or bigger aerodynamic size for deposition in central airways, or still bigger size for deposition in the mouth or the nasal cavity. The dry powder may also contain carrier particles to help the flow and dispersion of the therapeutic agent.

In another embodiment where the liquid nucleic acid-aminoglycoside complexes or compositions are administered to a patient in an aerosol inhalation device, the formulations of the invention are administered to a patient using a portable, hand-held, battery-powered device, such as the AERx device (Aradigm, Hayward, Calif.). Alternatively, the formulations of the instant invention could be carried out using a mechanical (non-electronic) device. Specific devices that may be used are disclosed in more detail in U.S. Pat. No. 5,544,646, issued Aug. 13, 19996 and U.S. Pat. No. 5,404,871, issued Apr. 11, 1995, both of which are incorporated herein by reference.

An aerosol may be created by forcing the nucleic acid-cationic aminoglycoside complexes or compositions through pores of a membrane which pores have a size in the range from about 0.25 to 6 microns. When the pores have this size, the particles which escape through the pores to create the aerosol will have a diameter in the range from about 0.5 to 12 microns. Drug particles may be released with an air flow intended to keep the particles within this size range. The creation of small particles may be facilitated by the use of a vibration device which provides a vibration frequency in the range from about 800 to about 4000 kilohertz. Those skilled in the art will recognize that some adjustments can be made in the parameters such as the size of the pores from which drug is released, vibration frequency, pressure, and other parameters based on the density and viscosity of the formulation, keeping in mind that the object is to provide aerosolized particles having a diameter in the range from about 0.5 to 12 microns.

Formulations of the invention may include an amount of alveolar surfactant protein effective to enhance the transport of the complexes across the pulmonary surface and into the circulatory system of the patient (see for example U.S. Pat. No. 5,006,343, issued Apr. 9, 1991, which is incorporated herein by reference).

The subject formulation may be provided as a low viscosity liquid formulation so that the formulation can be forced out of openings to form an aerosol, e.g., using about 20 to 200 psi, having a particle size in the range from about 0.5 to 12 microns.

In one embodiment of the subject invention, a low boiling point, highly volatile propellant is combined with the nucleic acid-aminoglycoside complexes of the subject invention and a pharmaceutically acceptable excipient. The nucleic acid-aminoglycoside complexes may be provided as a suspension or dry powder in the propellant, or, in another embodiment, may be dissolved in solution within the propellant. Both of these formulations may be readily included within a container which has a valve as its only opening. Since the propellant is highly volatile, i.e., has a low boiling point, the contents of the container will be under pressure.

In accordance with another formulation of the subject invention, the nucleic acid-aminoglycoside complexes are provided as a dry powder by themselves, and in accordance with still another formulation, the nucleic acid-aminoglycoside complexes are provided in a solution formulation. The inhaler, be it for dry powder formulation or liquid formulation, may also have the means to control the inspiratory flow rate, the inspired volume or both to achieve targeted deposition in the desired parts of the respiratory tract.

Any formulation which makes it possible to produce aerosolized forms of nucleic acid-aminoglycoside complexes which can be inhaled and delivered to a patient via the intrapulmonary route can be used in connection with the present invention.

In preparing the compositions in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs, and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated and enteric-coated by standard techniques.

For parenteral application by injection, preparations may comprise a pharmaceutically acceptable form of the nucleic acid-aminoglycoside complexes in an appropriate solution. Injectable suspensions may also be prepared using appropriate liquid carriers, suspending agents, agents for adjusting the isotonicity, preserving agents, and the like. Actual methods for preparing parenterally administrable compositions and adjustments necessary for administration to subjects will be known or apparent to those skilled in the art and are described in more detail in, for example, Remington's Pharmaceutical Science, 15th Ed., Mack Publishing Company, Easton, Pa. (1980), which is incorporated herein by reference.

For topical administration, the carrier may take a wide variety of forms depending on the preparation, which may be a cream, dressing, gel, lotion, ointment, or liquid.

Suppositories are prepared by mixing the liposome with a lipid vehicle such as theobroma oil, cacao butter, glycerin, gelatin, or polyoxyethylene glycols.

An effective amount of composition to be employed therapeutically will depend on a variety of factors, for example, upon the therapeutic objectives, the route of administration, the condition of the patient, and the like. Accordingly, it will be necessary for the clinician to titer the dosage and modify the route of administration as required to obtain the optimal therapeutic effect.

Additionally, the nucleic acid-aminoglycoside complexes according to the present invention may be administered in vivo in combination with other medicaments suitable for use in treating a particular disorder. For example, if the nucleic acid-aminoglycoside complexes contain a “suicide gene” which renders targeted cells susceptible to a particular drug, it may be desirable to coadminister the nucleic acid-aminoglycoside complexes in with the drug. In those embodiments of the subject complexes employing a liposome for encapsulating the nucleic acid, the drug may be, but need not be, also liposomally encapsulated.

Dosing

The doses of gene therapies will depend on a variety of factors including, but not limited to, the activity of the particular genes, the particular genetic defect or disease being treated, ancillary or concomitant illnesses or maladies, the type and amount of nucleic acid used, the desired level of gene expression, the balance of safety and efficacy and convenience and cost to the patient. By way of example and not limitation, for genes producing highly active compounds or for local applications, the doses can be in the microgram ranges. The maximum doses of orally administered therapeutics may be in the gram ranges, whereas, for example, inhalation doses are usually limited to the tens of milligrams per day range. These doses are exemplary only and of course may vary as required.

The nucleic acid-aminoglycoside complexes of the subject invention may be used for in vitro, in vivo and ex-vivo transfection into targeted cells. The targeted cells can be any cells. Oftentimes, the cell will have a cellular membrane comprised of a lipid bilayer, however, other times such a lipid bilayer will not be a requirement. Generally, targeted cells include eukaryotic cells, and typically mammalian cells, more typically murine or human cells.

If transfection is effected in vitro, a suitable amount of the subject nucleic acid-aminoglycoside complexes will be added to a cell culture medium containing the targeted cells. A suitable amount of the subject nucleic acid-aminoglycoside complexes composition may range from about 0.01 μg to about 25 μg per 10⁶ cells. Those of skill in the art will realize, however, that this amount may vary, depending upon factors such as the lability of the particular targeted cell, its resistance to transfection, the size of the particular nucleic acids, whether the nucleic acids are further encapsulated in a liposome, the activity of the particular gene, the desired level of gene expression, and the like.

The resulting transfected cells may be used for various applications. For example, the cells may be used to express a polypeptide encoded by the incorporated nucleic acids, e.g., a desired mammalian gene product. Also, if the incorporated nucleic acids result in the cells expressing a particular genetic defect, the cells may be used as models for studying the efficacy of proposed therapies for the particular genetic defect. Alternatively, if in vitro transfection results in the incorporation of genes which compensate for some genetic defect, or which encode a moiety such as an antisense RNA, ribozyme, or therapeutic protein, these cells may be administered to a host in need of genetic therapy (see for example U.S. Pat. No. 5,399,346, the disclosure of which is herein incorporated by reference).

If the nucleic acid-aminoglycoside complexes are to be used in vivo, they are thus administered to a host in need of such treatment. Another variation on in vivo use is for the generation of genetic defects, e.g., transgenic or “knock-out” mice which are useful in the study of disease. An example of treatment in a patient is when a DNA construct encoding the CFTR gene is complexed with an aminoglycoside such as tobramycin (and which may be encapsulated in a liposome) as described above and combined with the target cells of a patient suffering from cystic fibrosis. In addition to the above described therapeutic effect that corrects the cystic fibrosis deficient gene, the aminoglycoside used in combination with the nucleic acid may provide further therapeutic relief, i.e., bacteriostatic or bactericidal effects, as mentioned above, in particular for the treatment of gram-negative bacterial infections in this patient population.

The efficiency of in vivo or in vitro transfection may be measured by standard methods (see Sambrook et al. (1989), Molecular Cloning: a Laboratory Manual, Second Edition, Cold Spring Harbor Laboratory, Cold Spring Harbor, N.Y.). For example, the expression of genes encoded on an aminoglycoside complexed DNA construct transfected into cells in vitro can be studied by Northern blotting or RNA PCR to measure production of RNA transcripts, and by Western blotting, immunoprecipitation, and in situ immunohistochemistry to detect and measure protein production. Integration of the DNA into the host cell chromosome can be determined by PCR or by Southern blotting. The same methods are used to determine whether tissue treated in vivo contains transfected genes, or is expressing gene products of the transfected genes. This is preferably carried out on a biopsy sample of the tissue of interest.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g., amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

Preparation of Nucleic Acid-Aminoglycoside Complexes

Plasmid DNA and the three different prototype aminoglycosides (neomycin, gentamycin, tobramycin) were diluted to 2× the final desired concentration in separate vials. In formulations with a molar excess of DNA, the diluted aminoglycoside solution was added to the DNA using a pipette with light vortexing. In formulations with a molar excess of aminoglycoside, the DNA was added to the aminoglycoside solution with light vortexing. The formulations were allowed to rest for about 30 minutes before characterization.

### Example 2

Effectiveness of Complexing Nucleic Acids and Aminoglycosides

Complexes of Nucleic Acid-Aminoglycoside were prepared according to the above described method and then analyzed using agarose gel electrophoresis to verify that the nucleic acids were being complexed with the aminoglycosides.

Specifically, complexes having varying doses of DNA to three different aminoglycosides were prepared such that plasmid DNA was complexed with 5-100 mM of Neomycin, Tobramycin and Gentamicin. The results showed a dose-dependant increase in gel retardation, suggesting that the DNA was getting complexed with the aminoglycoside.

### Example 3

Stability of Nucleic Acid-Aminoglycoside Complexes

Complexes of Nucleic Acid-Aminoglycoside were prepared according to the above described method and then challenged with the endonuclease Dnase I.

The results indicated that the DNA complexation with the aminoglycosides were resistant to, or have conferred stability against, nuclease degradation.

### Example 4

In Vitro Transfection

Complexes of Nucleic Acid-Gentamicin were prepared according to the above described method and the transfection efficiencies thereof were evaluated in A-549 cells.

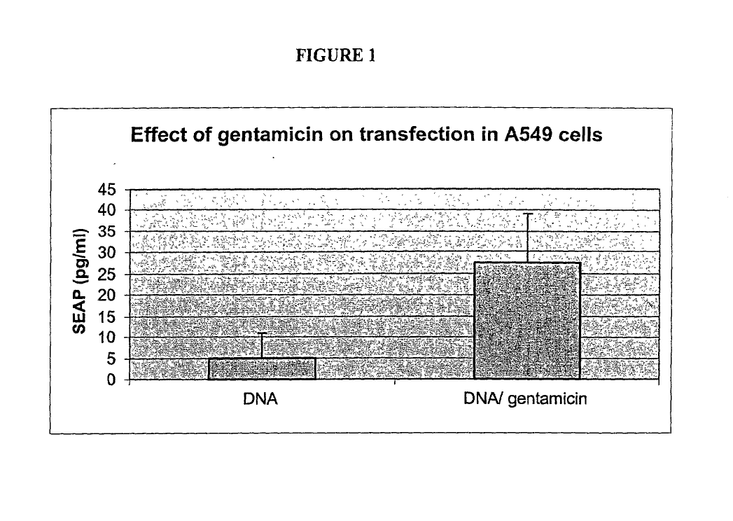

The results are shown in FIG. 1 and indicate that the complexes showed an increase in transfection efficiency compared to naked DNA.

The instant invention is shown and described herein in what is considered to be the most practical, and preferred embodiments. It is recognized, however, that departures may be made therefrom, which are within the scope of the invention, and that obvious modifications will occur to one skilled in the art upon reading this disclosure.

What is claimed is:

1. A composition for transfecting a cell with a nucleic acid, comprising:
a nucleic acid; and
a cationic aminoglycoside;
wherein said nucleic acid is condensed by interaction with said cationic aminoglycoside.

2. The composition according to claim 1, wherein said cationic aminoglycoside is bacteriostatic, and has an average molecular weight in a range of from 300 Daltons to about 800 Daltons.

3. The composition according to claim 1, further comprising water, wherein the composition has a physiological pH.

4. The composition according to claim 1, wherein said cationic aminoglycoside is selected from the group consisting of Gentamicin, Tobramycin, Amikacin, Streptomycin, Neomycin, Sisomicin and Netilmicin.

5. The composition according to claim 1, wherein said nucleic acid encodes a biologically active amino acid sequence.

6. The composition according to claim 1, wherein the composition is aerosolized and comprised of aerosol particles having an aerodynamic diameter in a range of from about 0.5 micrometer to about 12 micrometers.

7. The composition according to claim 1, wherein said nucleic acid is an antisense oligonucleotide.

8. The composition according to claim 1, wherein said nucleic acid is a high molecular weight polynucleotide comprising at least one coding region.

9. The composition according to claim 6, wherein the aerosol particles have an aerodynamic diameter in a range of from about 2 micrometers to about 6 micrometers.

10. The composition according to claim 1, wherein the composition is characterized by an ability to transfect human cells with an efficiency of 200% more as compared to transfer human cells about the cationic aminoglycoside.

11. The composition according to claim 1, wherein said composition is formulated in a pharmaceutical formulation designed to be administered to said cell by a means selected from the group consisting of pulmonary, parenteral, oral, nasal, intraperitoneal, intraocular, intracranial, suppository, dermal, transdermal and buccal.

12. The composition according to claim 1, wherein said composition further comprises at least one functional group, wherein said functional group is selected from the group consisting of targeting moieties, nuclear localization peptides and endosomolytic peptides.

13. The composition according to claim 1, wherein said composition further comprises at least one therapeutically acceptable lipid.

14. The composition according to claim 13, wherein said therapeutically acceptable lipid comprises a liposome encapsulating said nucleic acid.

15. The composition according to claim 1, wherein said condensation comprises a reduction of about 10³ to about 10⁶ in the physical volume of said nucleic acid.

16. A method of transfecting a cell comprising the steps of:
(a) contacting a composition comprising a nucleic acid and a cationic aminoglycoside with a cell; and
(b) allowing said composition to remain in contact with said cell for a period of time and under conditions such that said nucleic acid enters the cell.

17. The method according to claim 16, wherein said nucleic acid is condensed by the interaction with said cationic aminoglycoside.

18. The method according to claim 17, wherein said condensation comprises a reduction of about 10³ to about 10⁶ in the physical volume of said nucleic acid.

19. The method according to claim 16, wherein said nucleic acid encodes a biologically active amino acid sequence which is therapeutically effective.

20. The method according to claim 19, wherein the contacting is carried out by aerosolizing the formulation and inhaling aerosol into a patient's lungs.

21. The method according to claim 20, wherein said the aerosol has particles with an aerodynamic particle size in a range of from about 2 micrometers to about 6 micrometers.

22. The method according to claim 21, wherein said cationic aminoglycoside is selected from the group consisting of Gentamicin, Tobramycin, Amikacin, Streptomycin, Neomycin, Sisomicin and Netilmicin.

23. The method according to claim 22, wherein the composition further comprises at least one therapeutically acceptable lipid.

24. The method according to claim 23, wherein the lipid is a liposome encapsulating said nucleic acid.

25. The method according to claim 16, wherein said contact is accomplished in an environment selected from the group consisting of in vivo, in vitro and ex vivo.

26. The method according to claim 16, wherein the composition is aerosolized prior to contacting and the composition is brought in contact with the cell via pulmonary delivery of said composition to a subject.

27. A method of treatment, comprising:
aerosolizing a formulation comprising a nucleic acid and a cationic aminoglycoside to create aerosol particles having an aerodynamic diameter in a range of from about 0.5 micrometers to about 12 micrometers;
inhaling the aerosol into a patient's lungs; and
allowing the inhaled aerosol to contact cells for a period of time and under conditions such that the nucleic acid transfects the cells and expresses a therapeutically effective amount of a biologically active amino acid sequence.

28. The method according to claim 27, wherein the aerosoling in carried out by forcing said composition through pores of a membrane, wherein said aerosol has a particle size ranging from about 2 to about 6 microns.
